(19) United States
(12) Patent Application Publication
(10) Pub. No.: US 2022/0270731 A1
(43) Pub. Date: Aug. 25, 2022

(54) Structural Coupling Closed-Loop Feedback Remote Monitoring System and Method

(71) Applicants: Florida Institute for Human and Machine Cognition, Inc., Pensacola, FL (US); Monash University Malaysia SON BHD, Selangor Darul Ehsan (MY)

(72) Inventors: Nik Nailah Abdullah, Petaling Jaya (MY); Anil K. Raj, Pensacola, FL (US); Sergey V. Drakunov, Daytona Beach, FL (US); William J. Clancey, Pacific Grove, CA (US); David Steinhaus, Minneapolis, MN (US)

(73) Assignees: Florida Institute for Human and Machine Cognition, Inc., Pensacola, FL (US); Monash University Malaysia SDN BHD, Selangor Darul Ehsan (MY)

(21) Appl. No.: 17/399,128

(22) Filed: Aug. 11, 2021

Related U.S. Application Data
(60) Provisional application No. 63/152,404, filed on Feb. 23, 2021.

(51) Int. Cl. G16H 20/10; G16H 40/63

(52) U.S. Cl. CPC G16H 20/10; G16H 40/63

(57) ABSTRACT

An individualized machine learning model embedded on a mobile health application, coupled to autonomous workflow agents systems to operate as a small artificial intelligence program. The machine learning algorithm learns from small datasets—making it possible to compute real-time data even on a low-grade smartphone device. It predicts early worsening outcomes by extracting patterns (features) that correlate with the individual baseline changes in heart failure status. The workflow agents support dynamic machine learning model deployment to different devices and configurations for real-time prediction and intervention. The different device configurations include patients who own only a smartphone device, patients who own a smartphone and wearable/biosensors, and patients who own a smartphone and who have an implanted cardiac device.

### CROSS-REFERENCES TO RELATED APPLICATIONS

This non-provisional patent application claims priority to a previously filed provisional patent application. The parent application was filed on Feb. 23, 2021, and was assigned U.S. Patent Application No. 63/152,404. The parent application listed the same inventors.

### STATEMENT REGARDING FEDERALLY SPONSORED RESEARCH OR DEVELOPMENT

Not applicable

### MICROFICHE APPENDIX

Not Applicable

### BACKGROUND OF THE INVENTION

### 1. Field of the Invention

The present invention pertains to the field of medicine. More specifically, the invention comprises a closed-loop feedback software system embedded on a mobile application for medical therapy intervention in patients.

### 2. Description of the Related Art

The present invention is applicable to many different fields of endeavor. However, it is beneficial to the reader's understanding to describe the application of the invention to a specific field. Accordingly, this disclosure describes the invention's use in the field of medical diagnostics and intervention. The reader should bear in mind, however that the invention has many applications beyond this specific example.

Many medical conditions require frequency monitoring and intervention. Heart failure (“HF”) is a good example of such a condition. Heart failure is a condition whereby the heart has a reduced ability to pump an adequate supply of blood to meet the body's needs. As HF progresses and the heart weakens, reduced blood flow causes organ function impairment, requiring repeated hospitalization. The monitoring and treatment of heart failure is a good application for the present invention.

Heart failure has been described as a global pandemic, affecting about 26 million people worldwide (Ponikowski et.al., 2014). In Malaysia, for example, HF prevalence is estimated to be 1 to 2 percent of the entire population (3-20 cases per 1,000). Ten percent of these HF patients are more than 65 years old (100 cases per 1,000). Acute hospital admission due to HF accounts for 10% of all the hospitalizations in Malaysia. In addition, 25% of those HF patients who are hospitalized are re-hospitalized within thirty (30) days. The estimated overall cost of treating HF in Malaysia in 2014 was 194 million U.S. Dollars (approximately MYR 827, 507, 000.00) (Cook et.al., 2014). This cost represents approximately 1.8% of total health expenditure, with 3.6% of Malaysia's GDP being spent on healthcare.

In Southeast Asia, heart failure presents at a younger age (median of 54 years) compared with patients living in the United States of America (median of 75 years). Furthermore, patients in Southeast Asia are more likely to present with more clinical features, incur longer hospital stays, and suffer from higher in-hospital mortality (Lam, 2015).

Heart failure in the United States is an equally serious problem—affecting 6.5 million Americans at the time of this disclosure. It is now implicated in one in nine deaths in the United States and at least 20% of all hospitalizations among persons older than 65 (Savarese & Lund, 2017). The treatment of heart failure in the U.S. costs 30.7 billion U.S.D. annually, a figure forecasted to increase to 70 billion U.S.D. by 2030 (Agbor et al, 2020).

In China, HF affects more than 4 million individuals, with approximately 500,000 new cases diagnosed every year (Huang et al, 2016; Huang et al, 2017). HF accounts for 20% of hospitalizations and 40% of deaths due to cardiovascular diseases and is the second-highest cause of death in China. The mean annual healthcare cost per HF patient in China was 28,974 RMB (4,457 USD) and increased to 30,578 RMB (4,704 USD) in ≥65 age group. Hospitalization accounted for 65.6% of the total costs, whereas HF-related drug costs were only 8.2%. The mean hospital admission occurred 2.4 times per year. The all-cause readmission rate was 14.8% in 30 days and up to 59.0% within a single year. The mean hospital stay for HF patients was 30 days in one year; approximately >70% of hospitalizations were due to HF. Besides, per a global assessment, the total direct and indirect costs associated with HF annually were estimated at 5.4 billion U.S.D. in 2012 for China. With the increasing prevalence of coronary artery disease, hypertension, and an aging population, incidence, and prevalence of HF in China are projected to increase further, similar to many other low- and middle-income countries (Yu et al, 2019).

Due to the progressive nature of HF, it can lead to acute decompensated HF (“ADHF”). ADHF is defined as the sudden or gradual onset of the signs or symptoms of HF requiring unplanned office visits, emergency room visits, and hospitalization, which often leads to frequent re-hospitalization.

The frequent hospitalization of patients for the treatment of HF imposes a high burden on the health care system and resources. Thus, during an HF follow-up clinic session, health providers perform clinical assessments to determine the following: (1) whether a patient's health status demonstrates worsening outcomes (congestion); (2) whether the medical therapy being provided should be modified; and (3) whether the patient should be educated to monitor for symptoms, signs, fluid intake, and compliance to medical therapy.

Healthcare providers recognize the need for an effective outpatient monitoring and intervention system that helps keep HF patients out of the hospital by using patient self-monitoring. It is a demonstrated method to decrease patient admission (Lam, 2015) and reduce the economic burden on the health system (Lam, 2015). Nonetheless, there exist gaps in the needs in outpatient monitoring and intervention systems due to several reasons: (1) predicting reliably using a minimum set of data that can remotely verify whether a patient has indeed experienced worsening outcomes due to HF, so that appropriate intervention is taken, (2) the variations of predictors (i.e., parameters and variables) for different NYHA patient classification (class I-IV), and (3) delivery of adjusted medical therapy for patients who are located remotely from tertiary/primary care centers—a real problem in developing countries.

Chronic heart failure (CHF), otherwise known as congestive heart failure or simply “heart failure,” is an ongoing inability of the heart to pump enough blood through the body to ensure a sufficient supply of oxygen. The causes of the condition vary, but some of the most common risk factors include old age, diabetes, high blood pressure and being overweight. The condition affects the lower chambers of the heart, known as the right and left ventricles. The left ventricle pumps blood around the body, supplying it with oxygen. Chronic heart failure occurs when the heart cannot adapt to the body's changing need for oxygen, for example when exercising or climbing the stairs.

There are different types of chronic heart failure, classified according to how the heart reacts when it pumps. The two main types are:

(1) Heart failure with reduced ejection fraction: This type is also called systolic heart failure or HFrEF, and occurs when the heart is too weak and doesn't squeeze normally; and

(2) Heart failure with preserved ejection fraction: This type is also called diastolic heart failure or HFpEF, and occurs when the heart is too stiff and does not fill with blood normally.

Acute heart failure (“AHF”), also known as acute decompensated heart failure (“ADHF”) or cardiac failure, is not a single disease entity, but rather a syndrome of the worsening of signs and symptoms reflecting an inability of the heart to pump blood at a rate commensurate to the needs of the body at normal filling pressure (Teerlink, 2017).

Chronic heart failure (“CHF”) is generally a condition that develops gradually over time, whereas AHF, in most cases, occurs very suddenly and should be considered a medical emergency requiring immediate intervention. A CHF patient may transition into AHF progressively when there is poor management—most commonly a failure to modify the patient's lifestyle and/or a failure of effective medication titration. CHF patients therefore require a long-term chronic disease management system in order to provide timely intervention, especially for effective medication titration.

Acute heart failure may present in the patients as follows: (1) A pulmonary and/or peripheral edema, a term used to describe fluid overload in the organs or the body; and/or (2) A low output state—shock, a term used to describe “cold”—usually due to the heart's failure to pump adequately.

Some of the principles of management for acute heart failure are: (1) rapid recognition of the condition, (2) identification and stabilization of life-threatening hemodynamics, and (3) reliving clinical symptoms and signs. A clinician must guard against the progression of a patient toward acute heart failure. Thus, during an HF patient follow-up at the outpatient clinic, the goal of HF patient management is to make specific clinical assessments and decide whether a patient's condition is an indication of early fluid build-up in the body (such as fluid retention in the legs or abdomen). If the assessment results show the accumulation of fluid overload in the body, the patient warrants admission to decongest the fluids by medical therapy before the patient progresses to an acute decompensated heart failure state (“ADHF”).

The New York Heart Association has created a functional classification system for HF patients that is widely used. This classification system is presented in the following table:

TABLE ONE

| Class | Patient Symptoms |
|---|---|
| I | No limitation of physical activity. Ordinary physical activity does |
| | not cause undue fatigue, palpitation, dyspnea (shortness of |
| | breath). |
| II | Slight limitation of physical activity. Comfortable at rest. Ordinary |
| | physical activity results in fatigue, palpitation, dyspnea (shortness |
| | of breath). |
| III | Marked limitation of physical activity. Comfortable at rest. Less |
| | than ordinary activity causes fatigue, palpitation, or dyspnea. |
| IV | Unable to carry on any physical activity without discomfort. |
| | Symptoms of heart failure at rest. If any physical activity is |
| | undertaken, discomfort increases. |

In using this system, a patient is referred to as “NYHA Class n.” Typically, patients with NYHA Class III-IV are treated with implantable devices and a combination of medical therapy. Implantable devices include a Cardiac Resynchronization Therapy device (“CRT” or “pacemaker”) and an Implantable Cardiovascular Defibrillator (“ICD”). Some implantable devices include the functions of a CRT and ICD.

Many recent implantable devices include a monitoring and external communication feature. They collect data and can then wirelessly transmit that data to an external device. The present invention can take advantage of these functions for those patients having implants.

Different categories of HF patients have differing follow-up schedules such as (1) chronic but stable HF patients scheduled for visits every three months, every six months, or even once per year; (2) HF patients discharged from the hospital after a decongestion regimen followed up in two weeks or one month; (3) HF patients treated with implantable devices (CRTs, ICDs) seen in the device clinic in one month or six months.

During the period between follow-up visits, deterioration in the patient's HF status can occur rapidly. This is particularly true where the plaintiff does not comply with the treatment regimen—such as by failing to restrict fluid intake or closely monitor weight.

The present inventors have identified challenges in the existing chronic disease management systems, particularly for patients in remote areas such as exist in countries. The challenges identified are as follows:

(1) Decompensation of chronic HF usually presents with other signs of congestion and fluid retention, such as weight gain, exertional dyspnea, or orthopnea (difficulty sleeping at night). These symptoms and signs can begin weeks or days before the presentation; the onset occurs with abrupt cardiac output changes, which can delay or complicate timely intervention;

(2) HF patients require early recognition of worsening symptoms and signs so that proper self-management by titration of medication can be initiated (i.e., appropriate medication dosage adjusted, such as for diuretics or beta-blockers). However, recognizing whether symptoms and signs are due to worsening outcomes of the HF disease is in itself a challenge for the patient and for the clinicians to assess remotely; and (3) Clinicians require objective measurements to remotely determine whether a symptom (i.e., breathlessness) or a sign (i.e., swollen ankles) is indeed due to the progression of the HF disease or a typical variation in “baseline” symptoms for that patient as part of his/her HF disease.

Therefore, a need exists for improved methods in outpatient monitoring and intervention. The prior art systems typically address only the first of these three challenges. The present invention proposes an integrated solution that addresses all three of these challenges.

### BRIEF SUMMARY OF THE INVENTION

The present invention incorporates an individualized machine learning (ML) model embedded on a mobile health (mHealth) application (App), coupled to autonomous workflow agents systems. The ML algorithm learns from small datasets—making it possible to compute real-time data even on a consumer-grade smartphone device. It predicts early worsening outcomes by extracting patterns (features) that correlate with changes in the individual's baseline heart failure status. The workflow agents support dynamic ML model deployment to different devices configurations for real-time prediction and intervention. The different device configurations include patients who own only a smartphone device, patients who own a smartphone and wearable/biosensors, and patients who own a smartphone and also have an implanted device. Thus, the invention provides outpatient monitoring and intervention for the wider HF patient population. The workflow agents can be programmed to autonomously deliver medical therapy to patients with implantable devices and without implantable devices.

The invention opens a huge opportunity for HF patients and healthcare providers, in that it provides an affordable and accessible monitoring and intervention system that is well-suited to patients living in remote areas.

### BRIEF DESCRIPTION OF THE SEVERAL VIEWS OF THE DRAWINGS

FIG. 1 is a perspective view, showing a prior art smartphone.

FIG. 2 is a perspective view, showing a prior art smartphone.

FIG. 3 is a perspective view, showing a prior art blood pressure monitor.

FIG. 4 is a perspective view, showing a prior art pulse oximeter.

FIG. 5 is a schematic view, showing exemplary communications links.

FIG. 6 is a schematic view, showing the interaction among the components comprising the present invention.

### REFERENCE NUMERALS IN THE DRAWINGS

10 smartphone

12 interactive display

14 graphical user interface

16 microphone

18 digital camera

20 blood pressure cuff

22 interface module

24 pulse oximeter

26 cell tower

28 Internet

30 software agent

32 machine learning model

34 user

### DETAILED DESCRIPTION OF THE INVENTION

As discussed in the introduction, the present inventive method and system has many and diverse applications. The following detailed descriptions pertain to the field of medicine. The reader should bear in mind, however, that other applications exist and will be apparent to those skilled in the art.

The proposed invention provides a closed-loop feedback (“CLF”) software system embedded on a mobile application (“App”) for real-time tracking and monitoring of disease progression. A central goal of the invention is the prediction of early exacerbation and the provision of medical therapy intervention in HF patients.

The embedded software is preferably designed as a “small artificial intelligence (AI) program” so that the computation can take place at the edge (referring to the computation taking place on the native device rather than on a remote device or network-based service). Typical prior art systems perform most computations on large remote computing devices or network “cloud” services. The present invention allows the computation to take place on the native device, making the invention less reliant on Internet access and cloud computing. This makes remote monitoring and prediction accessible to a wider patient population.

The method uses patient-owned and carried devices (such as smartphones) to host the mobile App, which accesses real-time smartphone sensor data and provides a simple interface for users to input medical record data such as socio-demographic, comorbidity, drug dosage(s), and real-time subjective measurements (i.e., self-reported HF status and complaints such as symptoms, signs, and fluid intake) to log contextual information. The method extends the configured devices with the capability of real-time monitoring for individualized prediction of early exacerbation and medical therapy intervention by activation of the coupling function between appropriate machine learning (ML) models and workflow agents. The method applies to class I-IV HF patients. Thus, the inventive system and method can be used for HF patients with or without implantable devices and does not require a hospital patient management system (i.e., patient electronic medical records). The method can also be used in general for chronic disease management (e.g., diabetes).

A significant feature of the present invention is the rule activation function called the “coupling function” that joins the appropriate machine learning (“ML”) model with workflow agents to enable the prediction and intervention operations on different devices and configurations. The coupling function—inspired by the work of Maturana (1975, 1980)—describes how a single organism sustains its own survival. “Structural coupling” is the process by which several organisms establish working synergies after they have developed habits that correspond to the other's behavior. Of course, in the inventive method, the coupling function does not mimic the organism process but is used to describe the dynamic “joining” between the ML model and the workflow agents to enable prediction and intervention capabilities to be computed on the different device components and functionalities available.

The coupling method is a type of function (program) that supports the execution of the appropriate workflow automation by the use of software agents to configure the mobile App to the available device(s) parameters and, based on this, deploys the appropriate machine learning (“ML”) model. Once the ML model is deployed based on the recent configuration of devices—it leads to the execution of specific workflow agents for the patient cohort. The coupling function terminates the procedures after a decision is reached—a recorded action.

The coupling of the ML model with the workflow agents preferably includes a basic rules-activated function for appropriate workflow automation execution as described in the following:

(1) In the prediction of progression of a patient group P-Imp (a patient group with implanted devices), the machine learning targets include QRS (an electrocardiogram waveform measured by an implanted device) and HRV (heart rate variability as measured by an implanted device). Prediction will require correlation of features x, y. It then follows that patient group P-Imp will have a set of workflow automation A;

(2) In the prediction of progression of a patient group P˜Imp (without implants but only with a smartphone device) the machine learning targets include blood pressure, oxygen saturation, and edema. Prediction will require correlation of features x1,y1. It then follows that patient group P˜Imp will have a set of workflow automation B;

(3) In the prediction of progression of a patient group P-ExtDev (with a smartphone device and connected wearable device) machine learning targets include heart rate, oxygen saturation, and edema. Prediction will require correlation of features x1y1, x2y2. It then follows that patient group P-ExtDev group will have a set of workflow automation C.

The goal of the invention is not perfection. Rather, the goal is to provide “good enough” measurements and prediction for the patients that provide contextual feedback to doctors (via the mobile App), and to patients for screening, proper modification of management and timely intervention.

Thus, the inventive system and method defines the closed-loop feedback system to mean the capability provided by the machine learning model to predict reliably early exacerbation in an HF patient by correlating:

(1) objective parameters from device sensors (i.e., heart rate, heart rate variability, blood pressure) with

(2) the patient's subjective measurements (i.e., symptoms, signs), and

(3) contextual information (environmental state and stressors).

These operations are supported by the workflow software agents integrated within the App with different devices configurations. The medical therapy intervention can be programmed in various ways by using the inventive method. Examples include the following:

(1) Programmed to the guideline-directed medical therapy for HF as part of a component for the workflow agents to execute within a permissible range based on predicted values;

(2) Programmed so that the workflow agents deliver medical therapy intervention (i.e., titrate medication) within a permissible range in patients. Patients who do not have an implantable device would be aided by workflow agents to guide him/her in titrate medication in the intervention of medical therapy management;

(3) Programmed using a set of closed-loop control algorithm parameter ranges for each medical intervention in patients with implantable devices; and

(4) Programmed so that the workflow agent logs the outcome of the medical therapy intervention.

Medical therapy intervention that involves significant drug dosage modification and implant parameter adjustments would remain the purview of the clinician. The application of the inventive system and method is best explained using a specific example, which is provided in the following section.

Mr. Raman is a 48 year-old-man who lives in Kota Kinabalu, Malaysia. He has been living with HF disease for approximately one year. He is now being treated by Dr. Lee of the Hospital Jantung in Kota Kinabalu. Mr. Raman is a Class III NYHA patient. He last saw his physician about one month ago at the HF clinic at the Hospital Jantung.

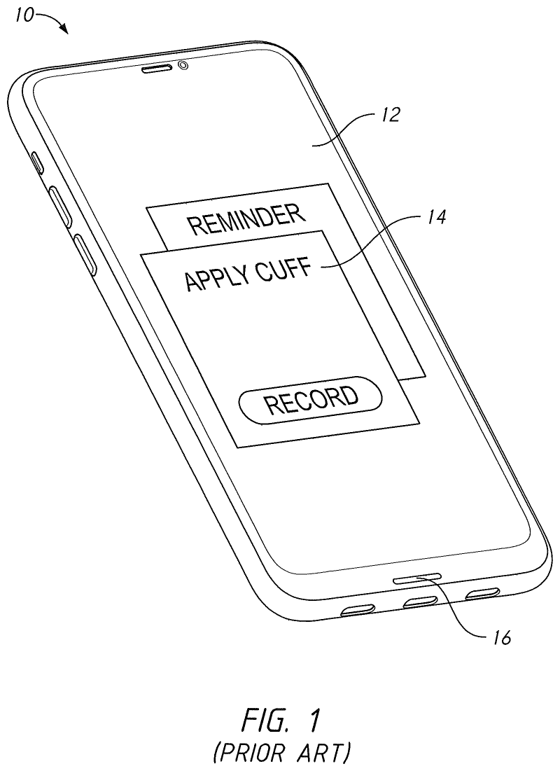

Mr. Raman owns and regularly uses a smartphone 10, such as shown in FIG. 1. This smartphone is a conventional device that contains an App forming a component of the present invention. Interface display 12 provides a touch-activated display. Graphical user interface 14 is presented by the App on interactive display 12.

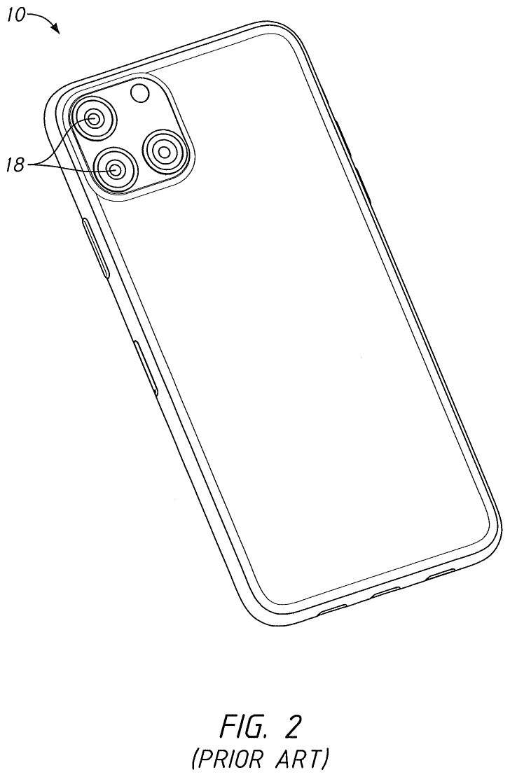

The smartphone includes various data input components, such as microphone 16 (as well as the interactive display which can be used to enter text, numbers, etc.). FIG. 2 shows smartphone 10 from the other side. The user will observe the presence of one or more digital cameras 18. These, along with internal motion sensors, can also be used to collect data for the App running on the device.

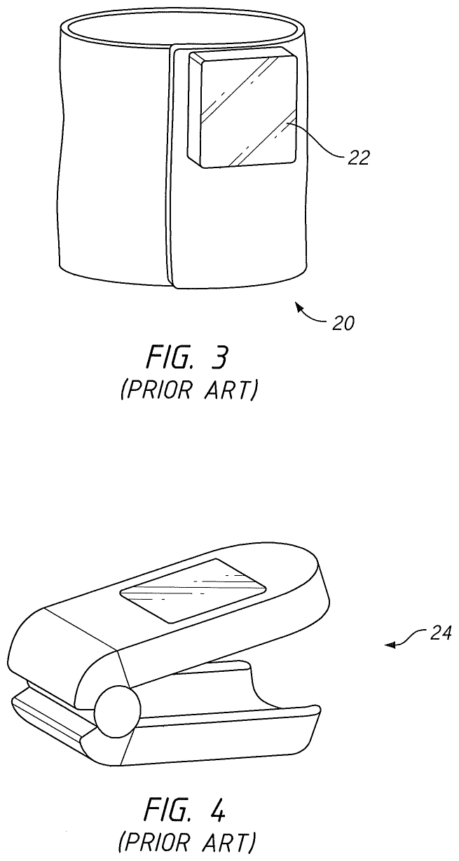

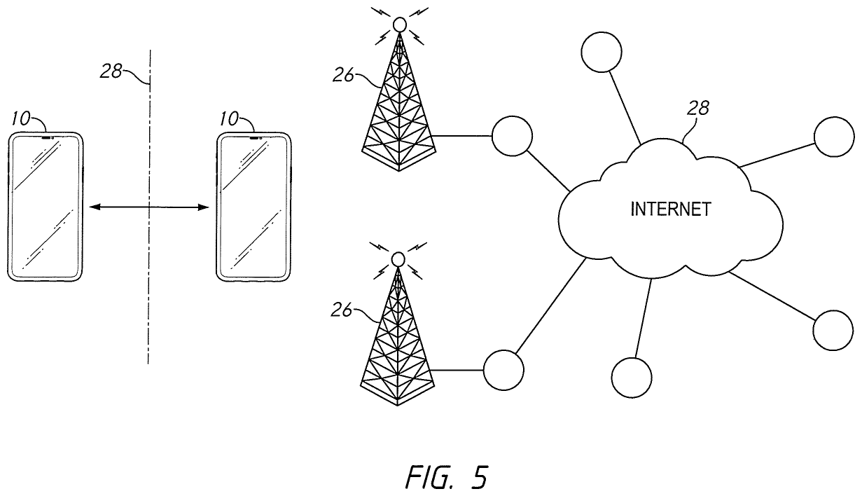

In addition to the smartphone itself, there now exist numerous external devices that can be used to collect medical data. FIGS. 3 and 4 show two such devices. FIG. 3 shows an existing blood pressure cuff 20. The patient can secure the cuff around his upper arm and initiate its operation using the smartphone. Interface module 22 collects blood pressure information and sends it wirelessly to the smartphone.

FIG. 4 shows an existing pulse oximeter 24 that is also wirelessly linked to the smartphone. The patient can insert his index finger into the pulse oximeter and initiate its operation using the smartphone. The pulse oximeter then collects oxygen saturation and pulse data and sends it wirelessly to the smartphone. The inventive App running on the smartphone collects this data and uses it in carrying out the present invention.

Most prior art medical applications assume full-time Internet connectivity for the smartphone. The smartphone in these applications acts as a data collection device. Large amounts of data are transmitted to an external computing device and most of the computations are done remotely. However, this configuration does not work well in areas where Internet connectivity is ether unavailable, sporadic, or impractically expensive.

FIG. 5 illustrates one scenario where Internet connectivity is limited. Internet 28 provides a connection between millions of nodes. Some of these nodes provide wireless connectivity via cell towers 26. Smartphone 10 is owned by a user such as Mr. Raman. In some periods during the day he is inside communication limit 28 and his phone can exchange data using a wireless connection. At other times his phone is outside the communication limit and must collect data and wait for an opportunity to transmit and receive. The present invention can be configured for use in environments with sporadic network communications.

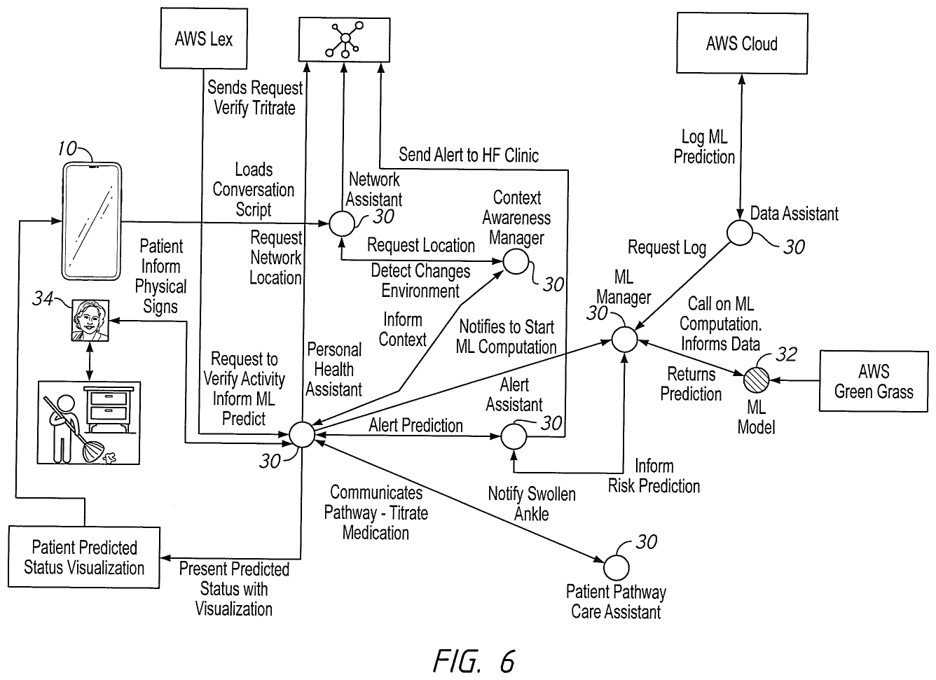

In this example Mr. Raman is home at noon, helping his wife do house chores. He has his smartphone in his possession. The Smartphone is running the inventive App, which includes several significant software agents. FIG. 6 graphically depicts the inventive system, along with the agents. Mr. Raman is user 34. The App is running on smartphone 10. The first software agent, known as the Context-Aware Manager agent, detects that he is about to do house chores and notifies the Personal Health Assistant agent. The Personal Health Assistant agent in this example is known as “Maria.” Maria prompts Mr. Raman to confirm that he is about to perform the house chores. It asks Mr. Raman to carry the smartphone in his pants pocket while performing the chores. It then notifies the Machine Learning Manager agent (“ML Manager”) that then calls on the machine learning model (ML Model 32) to start computation in the background. Meanwhile, the necessary data required for the ML Manager agent is pooled by the Data Assistant agent.

While doing work, Mr. Raman notices that his ankle is beginning to swell. He takes out his smartphone and uses the inventive App to report the physical sign that his ankle is swollen and rate the severity of his swollen ankle. Maria then reminds him to snap a picture of his swollen ankle so that it can record and analyze his vital signs with this event. Maria then communicates with the Patient Pathway Care Assistant agent information concerning the rated severity of Raman's swollen ankle to get recommendations on the next steps. The Patient Pathway Care Assistant agent then informs that the next step would be to titrate medication.

The software application running on the smartphone can be said to receive a first set of inputs and a second set of inputs. The first set of inputs comes from internal sensors within the smartphone and external sensors linked to the smartphone. Examples of the first set of inputs include an internal accelerometer used to detect and quantify motion (such as performing housework), an internal position sensor (such as GPS and cell-based geolocation sensors), an external heart rate sensor in an implantable device, and an external blood pressure sensor in a cuff the user periodically puts in place. The second set of inputs comes from the user interface. The user can provide text or numerical inputs in response to a prompt (“Rate your degree of pain on a scale of 1 to 10”). The user can also be prompted to take a photo of a swollen area.

Once the inputs have been received by the software application and the predictive functions performed, Maria (the Personal Health Assistant agent) next guides Mr. Raman on titrating his medication. However, before Maria takes this step, it communicates with the HF clinic staff to verify the instructions. The HF Clinic staff verifies the instruction, and Mr. Raman then titrates his medication as instructed by Maria. Mr. Raman then stops his activity.

After two days—Maria reminds Mr. Raman to log whether his swollen ankle has subsided after the titrating of his medication. In this example Mr. Raman informs Maria that the swollen ankle did not subside. Using the new data provided with the other multivariate data collected, the ML Manager agent detects that Raman is at risk of impending exacerbation and notifies the alert to the ML Manager, which then communicates with the Alert Assistant agent. The Alert Assistant alerts the HF clinic staff, and the Personal Health Assistant, Maria. Maria then informs Mr. Raman of his current health status and informs him that they have alerted the HF clinic staff. The personnel at the HF clinic staff will generally be referred to as the “monitoring clinician.” This term may encompass more than one person. As an example, the “monitoring clinician” can be one member of a group of physicians.

In the schematic depiction of FIG. 6, the reader will observe the interactions of the various software agents 30. The programmable agents are programmed with a specific role (i.e., “actors”), defined with a role and a Belief model. The belief model is programmed as conditional first-order logic. Whenever a certain fact (knowledge in the world) becomes true (for example, the patient has entered his/her input, the Belief then becomes true) a series of activities are carried out by the software agents as computer program functions. The software agents carry out specific activities by coordination with other agents and, in some scenarios, will collaborate to get specific data for the machine learning model to compute in real-time. The App provides a single point of entry for both user input and pooling and extracting device sensors, including external related health App or devices connected (i.e., wearable, biosensors). In the following sections an explanation is provided as to how the computation (i.e., procedure) takes place among the agents to form a workflow (i.e., different actors pass and process different data flows co-currently to achieve shared goals).

The following workflow automation preferably takes place in the inventive system:

(1) The Network Assistant agent finds out and verifies the location of the home setting (whether the patient is inside or outside of the home) and notifies the Context Awareness Manager agent of the location;

(2) The Context Awareness Manager agent requests the exact location and changes its states to its location (environment) indicated in the log that the patient is doing his usual household chores activity;

(3) This, in turn, triggers the states that the patient has a likelihood of symptoms and signs during this time;

(4) The Context Awareness Manager agent then informs the Personal Health Assistant agent (Maria) of the patient's context;

(5) The Personal Health Assistant agent requests the patient to verify the activity. When the state become true (patient is about to engage in the activity that he often experiences symptoms)—it notifies the ML Manager agent;

(6) Whenever a sign or symptom is logged indicating the severity of more than 4 rating scales, and the location is above the knee, the Personal Health Assistant agent will communicate with the Pathway Care Assistant agent for guidelines;

(7) The Personal Health Assistant agent then informs the Medical Health Assistant agent to get verification of guidelines from the HF Clinic Staff; and

(8) Once verification is provided, the Personal Health Assistant agent assists the patient in titrating medication.

The Personal Health Assistant agent will be programmed with “awareness” of current standard of care guidelines to prompt the patient to get information on the outcome of the interventions.

The inventive system and method preferably also include guideline-based medical therapy intervention procedures. In this scenario, the system will consider external devices and a related Health App that can provide good enough quality parameters such as tracking of physical activity (steps, distance, etc.), and manually provided blood pressure, heart rate, weight and medication dosage. Thus, this scenario requires interaction between the two software agents—the Personal Health Assistant agent and the ML Manager agent.

The Personal Health Assistant agent depends on the ML Manager agent to provide the required parameters for the ML model deployed for the specific patient device. It then needs to interact with the patient to perform the required actions so that other types of objective measurements can be recorded manually, such as blood pressure, heart rate, weight, and medication dosage.

The ML model would need to be robust in order to function even when one of the vital parameters required for prediction is interrupted or otherwise unavailable. It needs to give good enough prediction that propels the mixed-intervention of the Personal Health Assistant agent and nurses/doctors to verify further.

Thus, some of the preferred major activities of the Personal Health Assistant agent, which is programmed to the guideline medical therapy are as follows:

(1) It would communicate with patients, either when the patient self-reports, according to a pre-scheduled time, or when it detects changes in the activity of patients;

(2) It refers to the Clinical Pathway Care Assistant agent to get the instruction when a specific pre-defined event becomes true;

(3) It then instructs patients on what to do when a patient experiences specific symptoms following the pathway guideline;

(4) It then communicates with the Clinical Pathway Care Assistant agent to access the appropriate intervention guidelines on for the specific symptoms/signs;

(5) If titration of medication is indicated, it will first communicate with the Medical Health Assistant to ask for verification whether the instruction and dosage are correct for that patient's health status and co-morbidities;

(6) The Medical Health Assistant then requests that the HF clinician verifies or enters a dosage modification on the App, if required, based on the system prediction and objective clinical parameters;

(7) Once the HF clinician verifies the intervention, the Medical Health Assistant confirms with the Personal Health Assistant agent;

(8) The Personal Health Assistant agent engages in a dialog with the patient to guide the patient on how to the titrate the medication;

(9) It provides the ranking of the severity of signs to the Data Assistant agent;

(10) It will request from the patient subjective symptom reports following the medication titration, after an appropriate time, as indicated by the Clinical Pathway Assistant agent; and

(11) It logs the results using the Data Assistant agent.

The Personal Health Assistant agent has defined boundaries on what instructions or recommendations for the patients it can provide autonomously. These recommendations and actions must be logged for revision of the Clinical Pathway by cardiologists and pharmacists from time to time to ensure patient safety.

As the next preferred step in the workflow, the ML Manager agent would get pre-defined rules from the ML model on the objective parameters needed to predict near future outcomes using the logged data and its internal computational model. An overview of the preferred major activities conducted by the ML Manager agent is as follows:

(1) It will get baseline contextual information from the Data Assistant agent for a patient's normal blood pressure reading, heart rate, etc.;

(2) It will get the required objective parameters (e.g., heart rate readings, weight and weight changes, blood pressure) required by the ML model to estimate current and near future cardiac health;

(3) It requests that the Data Assistant records and pools the objective clinical parameters;

(4) It will call the ML model to compute and pass the data and correlate with the symptoms (if any), and the ranked physical signs (e.g., edema) and location (e.g., above the ankle, up to abdomen); and

(5) It verifies the predicted health status with the outcome of the medication titration. For example, if the ankle edema has not subsided and prediction shows an impending early onset of worsening outcome, it will proceed to alert the nurse/doctor to verify if an earlier follow-up is required. In circumstances where the predicted outcome is above a certain threshold for a specific sign or symptom, the Alerting Assistant agent will inform both the patient and doctor.

The Patient Pathway Care Assistant preferably accesses a set of prescribed recommendation and guidelines, which would be updated periodically by the HF clinic staff. The guideline-directed medical therapy would thus adjust but be tuned to the patient's baseline. The ML model would enable a safer drug dosage medication management by the agent and verified by HF clinicians because of the ability to track changes in the trends of vital signs after each recorded intervention.

The proposed method thus presents significant advantages, including the following:

(1) The method operates on a mobile App installed on a consumer-grade smartphone device to perform the required prediction and intervention operations. Thus, the method does not add additional cost to patients or healthcare providers by requiring the purchase of an additional device;

(2) The method can predict exacerbation by referring to individual's baseline for personalized prediction and without relying on clinical electronic medical record data;

(3) The method extends the capability of a consumer mobile App to function as a single element that processes input from all the required data needed for the complex computation. The data includes user input data (i.e., symptoms, signs, weight), including sensor data from the smartphone device itself (accelerometer sensors), implantable devices, or externally connected devices such as wearable or biosensors, or a third party related health that is required by the deployed ML model. These data are pooled and computed by the workflow agents to get the required quality parameters for the ML model to run the prediction in real-time, or to deliver services to the users. Thus, the method hides the system complexity from the users by the simple use of a mobile App user interface design, although the method is complex in the designing of the programmable workflow agents;

(4) The method enables a mobile App programmed as a simple tool for patient self-management, by allowing users to log-in their symptoms, or signs when requiring prediction or demand, or by prompting users to log-in symptoms or signs when the ML model detects early exacerbation. It functions just like a typical consumer mobile App to the users. It also allows healthcare provider to record and update basic medical record data via the App such as socio-demographic data, location, comorbidity and drug dosages. The method also allows doctors to specify target physical activity goal as part of patients' management plan. All these data used to adapt the precision in monitoring trends of disease progression and predicting early exacerbation;

(5) The method enables healthcare providers to add new actors (software agents) as part of the workflow automation for pharmacy, physical/occupational therapy, or the laboratory; and

(6) The method based on the principle of employing a small AI program will enable complex computation to run on native device, making it less dependent on cloud computing to perform real-time complex computation. Hence enabling wider access to HF patient population having limited or unstable Internet access.

The inventive system and method can be applied in a variety of medical settings. The following paragraphs provide additional information regarding these applications.

### EXAMPLE ONE—RURAL SETTING/DEVELOPING COUNTRY

In a remote and rural population in a developing country, the patient population often experience connectivity issues and have limited access to specialist healthcare providers. The inventive App is preferably configured to the smartphone device sensors and any related health App pre-installed on the patient's smartphone (i.e., Samsung Health App). Based on “good enough” parameters that can be provided by the patient's device sensors or related App, the workflow agents deploy the appropriate machine learning (“ML”) model for computation. Therefore, even if a patient only has a basic smartphone, the App can predict and trigger interventions with the health providers verifying the appropriate medical therapy (i.e., titrate medication) adjustment.

In a medical therapy intervention scenario, titration of medication based on individualizes patient's needs for diuretics and beta-blocker are programmed into the agents with instructions and recommended dosage, with a clinician-defined permissible range, that follow the programmed standard of care—medical therapy guidelines. The agents will automate presentation of the titration instructions when required in the workflow execution and communicate with the patient according to the recommended dosage and its instructions. The action recorded by the patients is logged and the parameters computed by the ML model are used to track changes in the individual patient's baseline.

In another scenario, hospitals managing the patients at primary care in an area far from the cardiac center can share information with the specialist to get verification on the recommended drug dosage modification programmed by the software agents.

### EXAMPLE TWO—DEVELOPED COUNTRY

The inventive app can be configured to interact with implantable devices, or with externally connected wearable devices or biosensors. In this scenario, the ML deployed would have available a greater quantity of higher quality parameters, and the software agents can be highly automated with recommended medical therapy based on guideline-directed medical therapy tuned to the patient's HF health status baseline. The clinicians can configure the software agents in the inventive App program with the permissible drug and/or auto adjustment of device parameters range that can be automated by the workflow agents based on the ML predicted values.

In a medical therapy intervention scenario, titration of medication based on individualized patient's needs for diuretics, beta-blocker, including Angiotensin Receptor-Neprilysin (ARNI) and sodium/glucose cotransporter 2 (SGLT2) inhibitors can be programmed for the agents for HF patients with implants. For example, as a beta-blocker is adjusted gradually, the ML would generate outcome predictions on a scheduled basis. Similarly, the action recorded by the patients logged and the required parameters computed by the ML model to trace changes in the patient's baseline.

In another scenario, a hospital wishes to include pharmacy, an agent programmed to interact and communicate directly with pharmacists, including update and dispensing of medicine at the appropriate pharmacy.

It is preferable for the embodiments of the present invention to utilize existing software capabilities where possible. Returning to FIG. 6, the reader will note the presence of blocks for “AWS Lex,” “AWS Cloud,” and “AWS Greengrass.” AWS Lex is a web-based ontology platform that allows the building of conversational interfaces into any App using voice and text. “AWS Cloud” is a web-based cloud computing platform. “AWS Greengrass” is a software platform that allows remote devices to run local computing, messaging, and data caching in a secure way during times when the remote device is not connected to the Internet. The exemplary blocks illustrated are products offered by AMAZON.COM of Seattle, Wash., U.S.A. The invention is by no means limited to the use of these particular platforms.

The preceding descriptions contains significant detail regarding the novel aspects of embodiments of the present invention. It should not be construed, however, as limiting the scope of the invention but rather as providing illustrations of the preferred embodiments of the invention. Thus, the scope of the invention should be fixed by the claims ultimately presented, rather than by the examples given.

Having described our invention, we claim:

1. A method for monitoring and treating heart disease in a patient having a smartphone with a set of internal sensors and a user interface configured for use by said patient, wherein said patient is within a communication limit for a first period of time and outside said communication limit for a second period of time, comprising:
(a) providing a software application running on said user's smartphone;
(b) said software application receiving a first set of inputs from said set of internal sensors within said smartphone and external sensors linked to said smartphone;
(c) said software application receiving a second set of inputs from said patient through said user interface;
(d) said software application predicting future changes in said heart disease within said patient by using a machine learning model to correlate,
(i) objective parameters within said first set of inputs,
(ii) subjective information from said patient in said second set of inputs,
(iii) contextual information determined from said first set of inputs; and
(e) said software application predicting said future changes while running on said smartphone while said patient is in said second period of time outside said communication limit.

2. The method for monitoring and treating heart disease in a patient as recited in claim 1, further comprising:
(a) providing said software application with a permissible dosage range for a medication being provided for treatment of said patient; and
(b) said software application calculating a recommended change to a dosage of said medication based on said future changes predicted by said software application.

3. The method for monitoring and treating heart disease in a patient as recited in claim 2, wherein:
(a) if said recommended change to said dosage results in a dosage that remains within said permissible dosage range, said software application providing a new recommended dosage to said user; and
(b) if said recommended change to said dosage results in a dosage that does not remain within said permissible dosage range, said software application advising said user to contact a monitoring clinician and provide said new recommended dosage to said monitoring clinician.

4. The method for monitoring and treating heart disease in a patient as recited in claim 3, comprising said software application automatically notifying said monitoring clinician when said smartphone is next within said communication limit.

5. The method for monitoring and treating heart disease in a patient as recited in claim 1, wherein said first set of inputs includes a motion sensor in said smartphone.

6. The method for monitoring and treating heart disease in a patient as recited in claim 1, wherein said first set of inputs includes a position sensor in said smartphone.

7. The method for monitoring and treating heart disease in a patient as recited in claim 1, wherein said set of external sensors linked to said smartphone includes a heart rate monitor.

8. The method for monitoring and treating heart disease in a patient as recited in claim 1, wherein said set of external sensors linked to said smartphone includes a blood pressure monitor.

9. The method for monitoring and treating heart disease in a patient as recited in claim 1, wherein said set of external sensors linked to said smartphone includes an implantable cardiovascular defibrillator.

10. The method for monitoring and treating heart disease in a patient as recited in claim 1, wherein said set of external sensors linked to said smartphone includes an implantable cardiac resynchronization therapy device.

11. A method for monitoring and treating heart disease in a patient having a smartphone with a set of internal sensors and a user interface configured for use by said patient, comprising:
(a) providing a software application running on said user's smartphone;
(b) said software application receiving a first set of inputs from said set of internal sensors within said smartphone;
(c) said software application receiving a second set of inputs from said patient through said user interface;
(d) said software application predicting future changes in said heart disease within said patient by using a machine learning model to correlate,
(i) objective parameters within said first set of inputs,
(ii) subjective information from said patient in said second set of inputs,
(iii) contextual information determined from said first set of inputs; and
(e) said software application predicting said future changes while running on said smartphone.

12. The method for monitoring and treating heart disease in a patient as recited in claim 11, further comprising:
(a) providing said software application with a permissible dosage range for a medication being provided for treatment of said patient; and
(b) said software application calculating a recommended change to a dosage of said medication based on said future changes predicted by said software application.

13. The method for monitoring and treating heart disease in a patient as recited in claim 12, wherein:
(a) if said recommended change to said dosage results in a dosage that remains within said permissible dosage range, said software application providing a new recommended dosage to said user; and
(b) if said recommended change to said dosage results in a dosage that does not remain within said permissible dosage range, said software application advising said user to contact a monitoring clinician and provide said new recommended dosage to said monitoring clinician.

14. The method for monitoring and treating heart disease in a patient as recited in claim 13, comprising said software application automatically notifying said monitoring clinician.

15. The method for monitoring and treating heart disease in a patient as recited in claim 11, wherein said first set of inputs includes a motion sensor in said smartphone.

16. The method for monitoring and treating heart disease in a patient as recited in claim 11, wherein said first set of inputs includes a position sensor in said smartphone.

17. The method for monitoring and treating heart disease in a patient as recited in claim 11, wherein said set of external sensors linked to said smartphone includes a heart rate monitor.

18. The method for monitoring and treating heart disease in a patient as recited in claim 11, wherein said set of external sensors linked to said smartphone includes a blood pressure monitor.

19. The method for monitoring and treating heart disease in a patient as recited in claim 11, wherein said set of external sensors linked to said smartphone includes an implantable cardiovascular defibrillator.

20. The method for monitoring and treating heart disease in a patient as recited in claim 11, wherein said set of external sensors linked to said smartphone includes an implantable cardiac resynchronization therapy device.
